(19) United States
(12) Patent Application Publication
(10) Pub. No.: US 2012/0190303 A1
(43) Pub. Date: Jul. 26, 2012

(54) CELL-PHONE COMPATIBLE WIRELESS STETHOSCOPE

(76) Inventor: Catherine Wong, Morristown, NJ (US)

(21) Appl. No.: 13/326,927

(22) Filed: Jan. 4, 2012

Related U.S. Application Data
(60) Provisional application No. 61/461,631, filed on Jan. 21, 2011.

(51) Int. Cl. H04W 80/00; A61B 7/04

(52) U.S. Cl. 455/41.2; 600/586

(57) ABSTRACT

A cell phone-compatible wireless stethoscope has an auscultation piece secured to an audio transmitter. The audio transmitter is configured to transmit audible biosignals detected by the auscultation piece. The signals may be transmitted via Bluetooth® to any Bluetooth®-capable cell phone and then transferred over a cellular network. The device may be used for remote medical examination and diagnosis.

### CROSS REFERENCE TO RELATED APPLICATION

This application claims priority of U.S. Provisional Patent Application No. 61/461,631, filed Jan. 21, 2011, which is hereby incorporated by reference.

### FIELD OF DISCLOSURE

The disclosed apparatus relates generally to medical devices, and more specifically to auscultation devices, such as stethoscopes.

### BACKGROUND

Physicians often use audible biosignals, such as cardiopulmonary sounds, to diagnose the medical condition of a patient. Audible biosignals are regularly detected using a stethoscope. In order to detect these signals, the physician needs to be in close physical proximity to the patient. Thus the physician and patient must travel to a common location before any diagnosis can occur. The travel may be problematic if the patient is, for example, in a remote or otherwise inaccessible location. Additionally, in an emergency situation such travel may waste valuable diagnostic time.

Telemedicine systems may be used to perform a remote diagnosis. Such systems, however, often require expensive specialized equipment. For example, a dedicated information network or a high speed internet connection may be required. Some networks may sacrifice audio quality for the sake of speed or efficiency, thereby increasing the possibility of miscommunication or misdiagnosis.

For example, in U.S. Pat. No. 6,533,736, issued to Moore, a wireless stethoscopic is disclosed having an auscultation piece removably secured to a hearing piece. Housed within the auscultation piece is a conventional radio frequency chip including a microphone that will enable transmission of radio frequency without the use of wires. A transmission system is housed within the auscultation piece, while the hearing piece includes a link manager receiver device for receiving the radio signals wirelessly from the transmission system and enabling sound to be heard via the hearing device from the auscultation piece. In U.S. Pat. No. 7,760,082, issued to Wong et al., a system and method are provided that to monitor life signs like heartbeat waveforms, body temperatures, indicating the health of a patient. The health of the Patient is defined by a set of known good spectra with deviations triggering alerts. A garment embedded with a piezoelectric material and a temperature sensor, when placed in contact with the body, captures acoustic waves from the heart and body temperature. Both sensors are connected to a garment-mounted module with a flexible printed antenna. Another module with reconfigured daughterboard software forms a bidirectional wireless data connection to a computer. A software program compares the received spectrum to its database spectrum based on a set of rules and alerts the user when it deviates. Also, the 3M® Littmann® Electronic Stethoscope provides on-board recording and playback capabilities, Bluetooth® technology to wirelessly transfer sounds to a computer system for further analysis.

These and other prior art systems provide some advantages, but at higher levels of structural complexity and concomitant increased costs. As a consequence, there is a need for a low cost device for transmitting audible biosignals from a patient to a physician, thereby allowing a prompt and reliable remote diagnosis.

### SUMMARY OF THE INVENTION

The present invention provides a stethoscope capable of transmitting biosignals over a telecommunications network. The stethoscope is formed from an auscultation head in a housing. The housing has a protruding stem and a diaphragm configured to receive audible biosignals, such as cardiopulmonary sounds. A wireless audio transmitter, such as a cellular telephone, is in communication with the auscultation head and may be used to transmit the audible biosignals to a remote location. The audio transmitter has a microphone located adjacent to the stem of the auscultation head by a tube, such that one end of the tube substantially surrounds the microphone and the other end substantially surrounds the stem.

### BRIEF DESCRIPTION OF THE DRAWINGS

These and other features and advantages of the present invention will be more fully disclosed in, or rendered obvious by, the following detailed description of the preferred embodiment of the invention, which is to be considered together with the accompanying drawings wherein like numbers refer to like parts and further wherein:

FIG. 1 is a perspective view of the wireless stethoscope formed in accordance with one embodiment of the present invention;

FIG. 2 is an exploded view of a wireless transmitter as shown in FIG. 1;

FIG. 3 is an exploded view of the auscultation head as shown in FIG. 1;

FIG. 4 is an exploded view of the coupling between the wireless transmitter of FIG. 2, and the auscultation head of FIG. 3.

### DETAILED DESCRIPTION OF THE INVENTION

This description of preferred embodiments is intended to be read in connection with the accompanying drawings, which are to be considered part of the entire written description of this invention. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures. It is noted that references in the specification to “one embodiment”, “an embodiment”, “an alternative embodiment”, etc., mean that the structures or procedures being described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, one of ordinary skill in the art would possess the knowledge to effect such feature, structure, or characteristic in connection with other embodiments whether or not explicitly described.

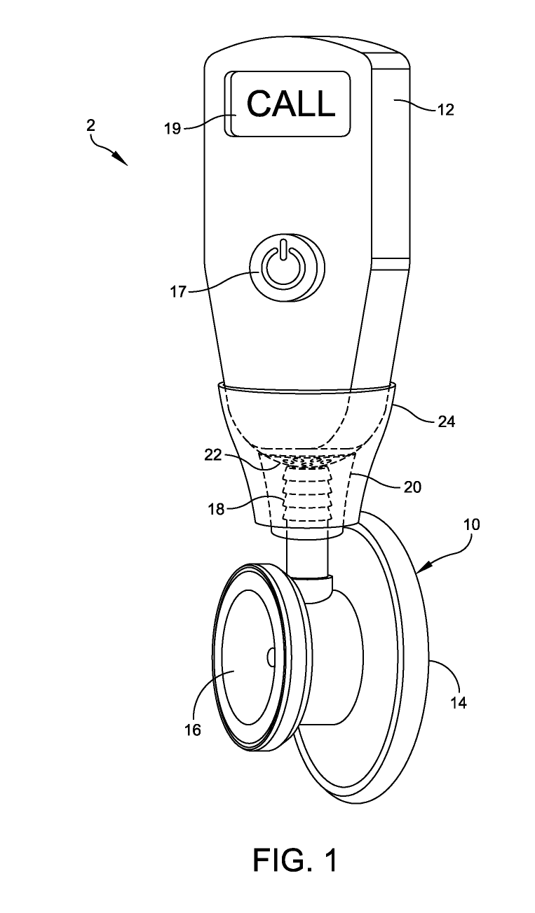

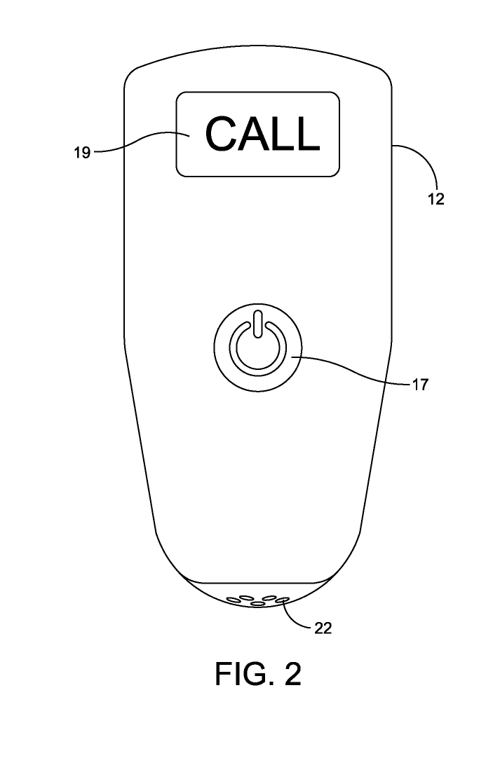

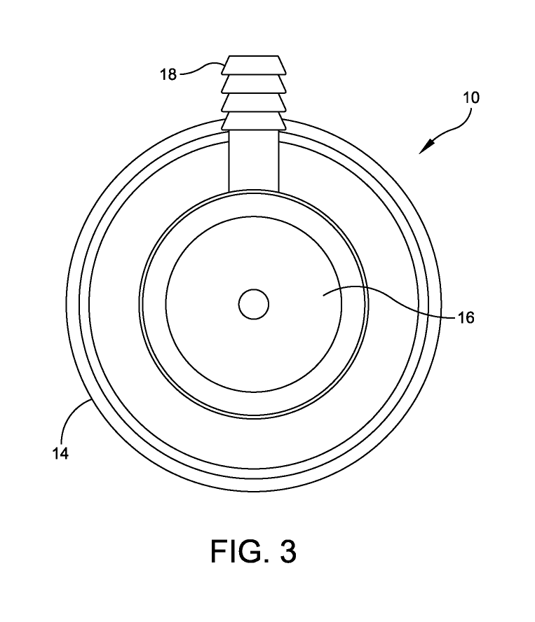

Referring to FIGS. 1, 2, 3, and 4, a wireless stethoscope 2 configured to transmit audible biosignals over a telecommunications network includes an auscultation head 10 and a wireless transmitter 12. More particularly, auscultation head 10 includes a diaphragm 14 configured to detect audible biosignals in a patient's body. These biosignals may be communicated to wireless transmitter 12, which in turn transfers them over a communications network, such as a cellular network, to a receiver (not shown), such as a cell phone held by a physician. The physician may listen to the biosignals and render a diagnosis without coming into physical contact with the patient.

Auscultation head 10 may be any standard stethoscope head. In one embodiment, auscultation head 10 comprises a housing 16 with a protruding stem 18 and a diaphragm 14 vibratably mounted to an opening defined in housing 16. Diaphragm 14 is configured to be positioned against a patient's body to detect audible biosignals. The biosignals are communicated through housing 16 and stem 18 to wireless transmitter 12. These biosignals may include, but are not limited to, cardiovascular, cardiopulmonary, or respiratory noises.

Auscultation head 10 may be of any size sufficient to detect patient biosignals and may be made of metal or a suitable polymer. In one embodiment, auscultation head 10 is at least three to five inches long, and about three to five inches wide, and is often circular in shape. Of course, other shapes are usable for auscultation head 10 such as square, rectangular, triangular, elliptical, etc. Similarly, diaphragm 14 often has a circular or oval shape. Diaphragm 14 may comprise any thin material capable of vibrating to detect, transmit, or amplify sound, such as thin rubber, metal, or plastic.

Wireless transmitter 12 may be any device capable of wirelessly transmitting audio signals over a communication network, and may comprise any transmission device readily available on the market. In one embodiment, the transmitter is a telephone connected to a cellular network. Alternatively, the device may be a Bluetooth® transmitter. The Bluetooth® transmitter may be in communication with a Bluetooth® enabled cellular phone, which is in turn connected to a cellular network. The Bluetooth® transmitter may also be in communication with a base station or personal computer connected to the internet. In one embodiment, the Bluetooth® transmitter may be directly connected to a device on the physician's person. Such an embodiment may be effective in a clinic setting where the physician is moving about the facility to aid different patients. Wireless transmitter 12 may further comprise power button 17 and status indicator 19.

Wireless transmitter 12 may be sufficiently small and lightweight to fit comfortably in one hand. In one embodiment, the transmitter is three to six centimeters long, two to three centimeters wide, and ten to fifty grams in weight. In another embodiment where the wireless transmitter is a Bluetooth® transmitter in communication with a Bluetooth® receiver, the transmitter may have a communication distance of at least one to five feet. Other embodiments allow a greater or a lesser transmission range.

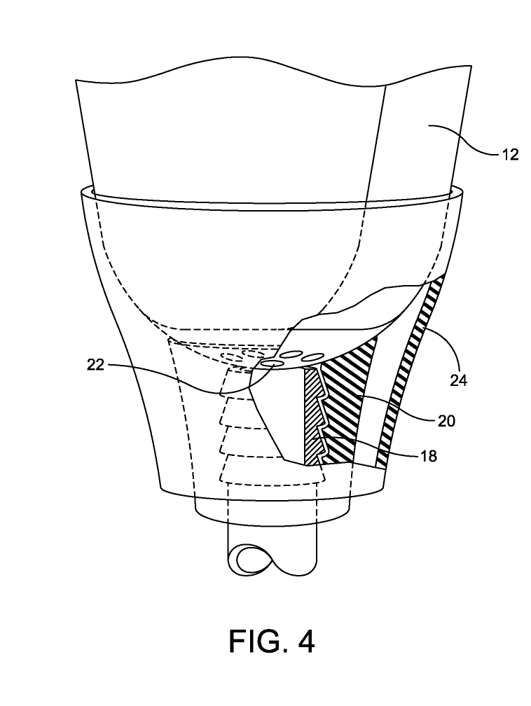

As shown in FIGS. 1 and 4, auscultation head 10 and wireless transmitter 12 may be coupled to each other using a hose 20. A first end of hose 20 is connected to stem 18 protruding from housing 16. A second end of hose 20 is secured in surrounding relation to microphone 22 connected to the audio transmitter 12. Audible biosignals may be communicated from auscultation head 10, through hose 20 secured to protruding stem 18, and into the microphone 22. In one embodiment, microphone 22 may be placed flush against stem 18. Alternatively, microphone 22 may be separated from the stem up to any distance that allows reliable communication of the biosignals.

In another embodiment, hose 20 may be one to four centimeters in length, with a radius of one to two centimeters. Stem 18 may be one to three centimeters long, with a radius of about 0.5 to one centimeter. Hose 20 may comprise any flexible or rigid material, such as rubber or plastic.

A second length of tube, such as heat shrink tubing 24, may surround hose 20, microphone 22, and stem 18. When heat is applied to heat shrink tube 24, the tube's circumference will shrink so as to form a tight seal around hose 20. This serves to reinforce the coupling between auscultation head 10 and wireless transmitter 12, and may also protect microphone 22 and stem 18 from moisture. In another embodiment, tube 24 is slightly longer than the interior of hose 20. For example, the heat shrink tube 24 may be about one to five centimeters in length, with a radius of about one to three centimeters.

In one embodiment, hose 20 or the heat shrink tube 24 may sound insulation, such as a foam strip, spray, or tape, that encapsulates the hose and coupling. This sound insulation may help to isolate microphone 22 and stem 18 from ambient noise. Such isolation often ensures that the only noises received by the microphone, and transmitted by wireless transmitter 12, are the audible biosignals communicated by auscultation stem 18. Such an embodiment may be useful, for example, in an emergency setting where the patient is surrounded by emergency personnel and sirens.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

What is claimed is:

1. A medical diagnostic apparatus, comprising:
an auscultation head configured to capture audible biosignals and having a housing, a diaphragm secured to said housing, and a stem protruding from the housing; and
a wireless transmitter in sound communication with the auscultation head and configured to transmit said audible biosignals from the auscultation head to a remote location, said wireless transmitter comprising a microphone secured to said stem by a first length of tube such that the microphone is substantially encompassed by a first end of the tube and the stem is substantially encompassed by a second end of the tube.

2. The apparatus of claim 1, wherein the audio transmitter is a cellular telephone.

3. The apparatus of claim 1, wherein the audio transmitter is a Bluetooth® transmitter.

4. The apparatus of claim 3, wherein the Bluetooth® transmitter is in communication with a cellular telephone and has a range of one to five feet.

5. The apparatus of claim 3, wherein the Bluetooth® transmitter is in communication with a Bluetooth® base station.

6. The apparatus of claim 5, wherein the Bluetooth® base station is a personal computer connected to the internet.

7. The apparatus of claim 1, further comprising a second tube surrounding the first length of tube, wherein said second tube is heat shrink tubing and forms a seal around microphone and the stem.

8. The apparatus of claim 1, wherein the first length of tube is substantially encapsulated by a sound resistant material.

9. The apparatus of claim 1, wherein the wireless audio transmitter comprises an elongated housing three and six centimeters long, and two to three centimeters wide.

10. The apparatus of claim 1, wherein the audio transmitter weighs between ten and fifty grams.

11. The apparatus of claim 1, wherein the auscultation head is circular and has a diameter between three and five inches.

12. The apparatus of claim 1, wherein the stem is cylindrical and has a radius of 0.5 to one centimeter.

13. The apparatus of claim 1, wherein the stem protrudes one to three centimeters out from the housing.

14. The apparatus of claim 1, wherein the auscultation head has a shape selected from the group consisting of square, rectangular, triangular, elliptical, and oval.
